(19) United States
(12) Patent Application Publication
(10) Pub. No.: US 2018/0222652 A1
(43) Pub. Date: Aug. 9, 2018

(54) ARTICLE FOR STORAGE OF THIN-FILM DRUG

(71) Applicant: Par Pharmaceutical, Inc., Chestnut Ridge, NY (US)

(72) Inventor: Craig BERRY, Westport, CT (US)

(21) Appl. No.: 15/890,724

(22) Filed: Feb. 7, 2018

Related U.S. Application Data
(60) Provisional application No. 62/456,336, filed on Feb. 8, 2017.

(51) Int. Cl. B65D 75/30; B65D 75/58; B65B 11/50; A61J 7/00; A61P 25/16; A61K 9/70; A61K 31/485

(52) U.S. Cl. CPC B65D 75/305; B65D 75/585; B65B 11/50; A61K 31/485; A61P 25/16; A61K 9/7007; A61J 7/0015

(57) ABSTRACT

The present invention describes an article for storage and delivery of a unit dosage form of a drug. The unit dosage form can be in the form of a thin-film or transdermal patch. The article can be used to facilitate removal of a unit dosage form of the therapeutic for those subjects having reduced dexterity.

### CROSS REFERENCE

This application claims the benefit of U.S. Provisional Application No. 62/456,336, filed Feb. 8, 2017, the entire content of which is incorporated herein by reference in its entirety.

### BACKGROUND

Thin-film drug products can be packaged as single dose films in peelable pouches for convenience, avoidance of contamination, and for preservation of drug integrity. Peelable pouches can be used to store therapeutics for various disorders, including neurodegenerative disorders and addiction, and for long-term birth control methods. However, if not opened properly, peelable pouches can lead to the tearing of the thin-film drug product, and can pose difficulties for users with compromised dexterity.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, the invention provides an article for storage of a therapeutic, the article comprising: a) a first flexible layer, wherein the first flexible layer comprises a right-tab and a backing layer; and b) a second flexible layer, wherein the second flexible layer comprises a left-tab and a cover layer, wherein the right-tab and the left-tab are adjacent and connected to each other along an axis, wherein the right-tab and the left-tab are in a common plane, and wherein the axis is weakened in comparison to the right-tab and the left-tab; and wherein the cover layer is overlaid on the backing layer to create an enclosure of a size to hold a unit dosage form of the therapeutic, wherein the cover layer and backing layer are sealed together along a perimeter of the enclosure, wherein a portion of the perimeter of the enclosure is adjacent to an end of the axis.

In some embodiments, the invention provides a method for storage of a therapeutic, the method comprising: a) inserting a unit dosage form of the therapeutic in a cavity; and b) sealing the cavity along a perimeter of the cavity to transform the cavity into an enclosure of size to hold the unit dosage form of the therapeutic; wherein the enclosure forms part of an article for storage of the therapeutic, wherein the article comprises: i) a first flexible layer, wherein the first flexible layer comprises a right-tab and a backing layer; and ii) a second flexible layer, wherein the second flexible layer comprises a left-tab and a cover layer, wherein the right-tab and the left-tab are adjacent and fastened to each other along an axis, wherein the right-tab and the left-tab are in a common plane, and wherein the fastening of the axis is weakened with respect to the right-tab and the left-tab; and wherein the cover layer is overlaid on the backing layer to create the enclosure of the size to hold the unit dosage form of the therapeutic, wherein the cover layer and backing layer are sealed together along a perimeter of the enclosure, wherein a portion of the perimeter of the enclosure is adjacent to an end of the axis.

In some embodiments, the invention provides a method of opening an article for storage of a therapeutic by a subject, the method comprising: a) gripping a left-tab and a right-tab of the article, wherein the article comprises: i) a first flexible layer, wherein the first flexible layer comprises the right-tab and a backing layer; and ii) a second flexible layer, wherein the second flexible layer comprises the left-tab and a cover layer, wherein the right-tab and the left-tab are adjacent and fastened to each other along an axis, wherein the right-tab and the left-tab are in a common plane, and wherein the fastening of the axis is weakened with respect to the right-tab and the left-tab; and wherein the cover layer is overlaid on the backing layer to create an enclosure of a size to hold a unit dosage form of the therapeutic, wherein the cover layer and backing layer are sealed together along a perimeter of the enclosure, wherein a portion of the perimeter of the enclosure is adjacent to an end of the axis; b) pulling apart the left-tab and the right-tab so that the left-tab and right-tab are no longer adjacent to each other; and c) peeling open at least part of the seal of the enclosure of the size to hold the unit dosage form of the therapeutic using the left-tab and the right-tab; wherein the peeling open of the enclosure provides an aperture that renders accessible the unit dosage form of the therapeutic to the subject.

In some embodiments, the invention provides a kit comprising: a) a package, wherein the package comprises: i) a first flexible layer, wherein the first flexible layer comprises a right-tab and a backing layer; and ii) a second flexible layer, wherein the second flexible layer comprises a left-tab and a cover layer, wherein the right-tab and the left-tab are adjacent and fastened to each other along an axis, wherein the right-tab and the left-tab are in a common plane, and wherein the fastening of the axis is weakened with respect to the right-tab and the left-tab; and wherein the cover layer is overlaid on the backing layer to create an enclosure of a size to hold a unit dosage form of a therapeutic, wherein the cover layer and backing layer are sealed together along a perimeter of the enclosure, wherein a portion of the perimeter of the enclosure is adjacent to an end of the axis; and b) a therapeutic enclosed in the enclosure.

In some embodiments, the invention provides a method of treating a condition, the method comprising administering to a subject in need thereof a unit dosage form of a therapeutic, wherein the unit dosage form of the therapeutic is housed in an article for storage of the unit dosage form of the therapeutic, wherein the article for storage of the unit dosage form of the therapeutic is opened by the subject, wherein the opening of the article for storage of the unit dosage form of the therapeutic by the subject comprises: a) gripping a left-tab and a right-tab of the article by the subject, wherein the article comprises: i) a first flexible layer, wherein the first flexible layer comprises the right-tab and a backing layer; and ii) a second flexible layer, wherein the second flexible layer comprises the left-tab and a cover layer, wherein the right-tab and the left-tab are adjacent and fastened to each other along an axis, wherein the right-tab and the left-tab are in a common plane, and wherein the fastening of the axis is weakened with respect to the right-tab and the left-tab; and wherein the cover layer is overlaid on the backing layer to create an enclosure of a size to hold the unit dosage form of the therapeutic, wherein the cover layer and backing layer are sealed together along a perimeter of the enclosure, wherein a portion of the perimeter of the enclosure is adjacent to an end of the axis; b) pulling apart the left-tab and the right-tab so that the left-tab and right-tab are no longer adjacent to each other; c) peeling open at least part of the seal of the enclosure of the size to hold the unit dosage form of the therapeutic using the left-tab and the right-tab; wherein the peeling open of the enclosure provides an aperture that renders accessible the unit dosage form of the therapeutic to the subject; and d) removing the unit dosage form of the therapeutic by transferring the unit dosage form of the therapeutic through the aperture to the mouth of the subject.

### BRIEF DESCRIPTION OF THE FIGURES

FIG. 1 provides an illustrative example of an article of the invention.

FIG. 2 provides an illustrative example of an article of the invention.

FIG. 3 provides an illustrative example of a side view of an article of the invention.

FIG. 4 provides an illustrative example of an opened article of the invention.

FIG. 5 provides an illustrative example of an opened article of the invention.

FIG. 6 provides an illustrative example of a side view of an article of the invention.

FIG. 7 depicts an illustrative example of a back view of an article of the invention.

FIG. 8 depicts a separated version of an article of the invention.

FIG. 9 depicts a separated version of an article of the invention.

FIG. 10 is an illustrative view of a machine and method for packaging an article of the invention.

FIG. 11 is an illustrative view of another machine and method for packaging an article of the invention.

### DETAILED DESCRIPTION

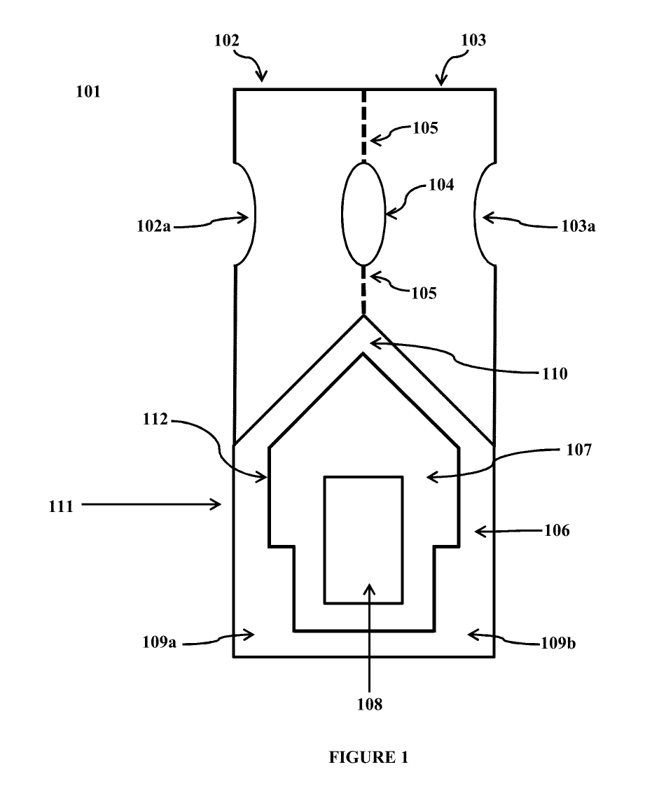

The present invention provides an article that can be used to, for example, package, store, and deliver a drug that is in the form of, for example, a transdermal patch or a thin-film. An article described herein can be a pouch that can be peeled or unsealed by a subject. FIG. 1 provides an illustrative example of an article of the invention 101. Non-limiting examples of materials that can be used in the manufacture of the article include aluminum, foil, olefin fibers, paper, plastic, polyester, polyethylene fibers, polyethylene, and terephthalate. The aluminum can be an aluminum alloy containing, for example, chromium, copper, iron, magnesium, manganese, silicon, titanium, or zinc.

FIG. 1 shows that the article contains a left-tab 102 and a right-tab 103, which are adjacent to each other. The left-tab 102 and right-tab 103 are used to grasp the article 101 by a user. The left edge of the left-tab 102 further contains a cut-out 102a, and the right edge of the right-tab 103 contains a cut-out 103a to facilitate gripping of the article by the user. The left-tab 102 and right-tab 103 are fastened to each other at a longitudinal axis 105. The longitudinal axis can, for example, be at a longitudinal media. The longitudinal axis 105 is perforated to allow for separation of the left-tab 102 from the right-tab 103 upon pulling of the left-tab 102 and right-tab 103 in opposing directions by the user so that the left-tab 102 and the right-tab 103 are no longer in a common plane. The left-tab 102 and right-tab 103 can contain a cut-out 104 along the longitudinal axis to facilitate separation of the left-tab 102 from the right-tab 103 when the user grabs the left-tab 102 and right-tab 103 using fingers of both hands. For example, the user can grasp the left-tab 102 using the index finger and thumb of the left hand, and grasp the right-tab 103 using the index finger and thumb of the right hand.

An enclosure 111 of a size to hold a therapeutic 108 in the form of, for example, a thin film, is attached to the left-tab 102 and the right-tab 103. The enclosure is formed by overlaying a first flexible layer 106 and a second flexible layer 107, and sealing the layers using an adhesive to create a cavity 112 for storage of the therapeutic 108. The adhesive used to seal the flexible layers can be, for example, ABS (acrylonitrile-butadiene-styrene), acetal, aclar, acrylic, acrylonitrile-methyl acrylate copolymer, APET, butyrate, cellulose acetate, cellulose acetate butyrate, cellulose triacetate, cellulose nitrate, CPET, epoxy, ethyl cellulose, EVA (ethyl vinyl acetate), EVOH (ethyl vinyl alcohol), fluorinated ethylene-propylene copolymer, hydroxyethyl cellulose, methyl cellulose, nylon (polyamide), pellethane, PET (polyethylene terephthalate), PETG (polyethylene terephthalate glycol), phenol-formaldehyde, polycarbonate, polyethylene, polyethylene resin, polyester-polyurethane resin, polyol-polyurethane resin, polyurethane, polyurethane foam, polyvinyl butyral, polyvinylidene chloride, vinyl chloride-acetate, polyethylene glycol, polyimide, polymethyl methacrylate, polypropylene, polystyrene, polyvinyl acetate, polyvinyl acetate-chloride copolymer polyvinyl chloride, polyvinyl acetal, polyvinylidene chloride, polyvinyl formal, polyalkylene terephthalate, polyvinyl alcohol, rubber, silicone, sodium carboxymethyl, tetrafluoroethylene, or vinyl resin.

The right-tab 103 is attached to the first flexible layer 106. For example, the right-tab 103 and the first flexible layer 106 can exist as a single article, with the right-tab 103 and the first flexible layer 106 being regions of the same article. The left-tab 102 is attached to the second flexible layer 107 similarly. When the user pulls apart the left-tab 102 and the right-tab 103, the left-tab 102 pulls the first flexible layer and the right-tab 103 pulls the second flexible layer in different directions. Thus, the seal 110 is unsealed to open the cavity 112 housing the therapeutic 108.

FIG. 1 further shows that the enclosure 111 can be sealed using a chevron-shaped seal 110, which is peeled by the user by pulling apart of the left-tab 102 and right-tab 103 to expose the therapeutic 108 to the user. The bottom of the article 101 contains wider seals 109a and 109b that can create resistance to prevent complete separation of the article, because the wider seals contain a greater amount of adhesive than do the other parts of the seal. The resistance can allow the therapeutic 108 to remain in the enclosure to facilitate removal by the user.

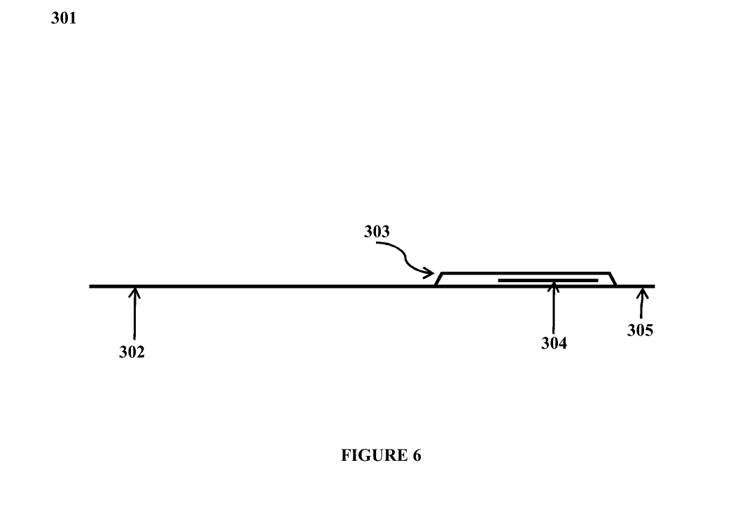

FIG. 6 depicts a side-view of an article of the invention 301. The left-tab 102 and right-tab 102 of FIG. 1 are shown as the substantially-flat portion 302. FIG. 6 further shows the enclosure 303 housing the therapeutic 304 in an article of the invention 301. The wider seals 109a and 109b of FIG. 1 are shown as the substantially-flat portion 305.

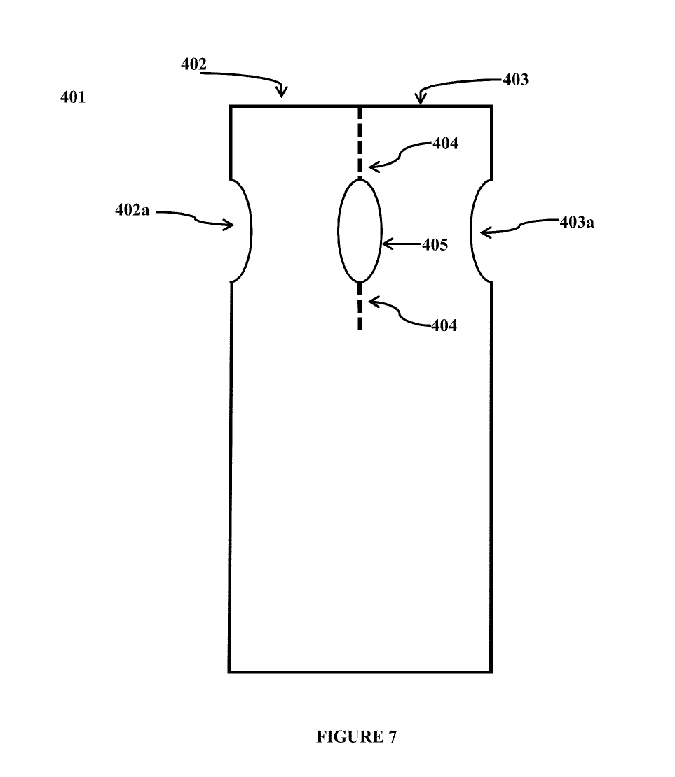

FIG. 7 depicts the back view of an article of the invention 401. FIG. 7 shows that the article contains a left-tab 402 and a right-tab 403, which are adjacent and fastened to each other. The left-tab 402 and right-tab 403 are used to grasp the article 101 by a user. The left edge of the left-tab 402 further contains a cut-out 402a, and the right edge of the right-tab 403 contains a cut-out 403a to facilitate gripping of the article by the user. The left-tab 402 and right-tab 403 are joined to each other at a longitudinal axis 404. The longitudinal axis can be, for example at a longitudinal median. The longitudinal axis 105 is weakened with respect to the right-tab and the left-tab to allow for separation of the left-tab 402 from the right-tab 403 upon pulling of the left-grip 402 and right-grip 403 in opposing directions by the user so that the left-tab 402 and the right-tab 403 are no longer in a common plane. The longitudinal axis 105 can, for example, be perforated. The left-tab 402 and right-tab 403 contain a cut-out 405 along the longitudinal axis to facilitate separation of the left-tab 402 from the right-tab 403.

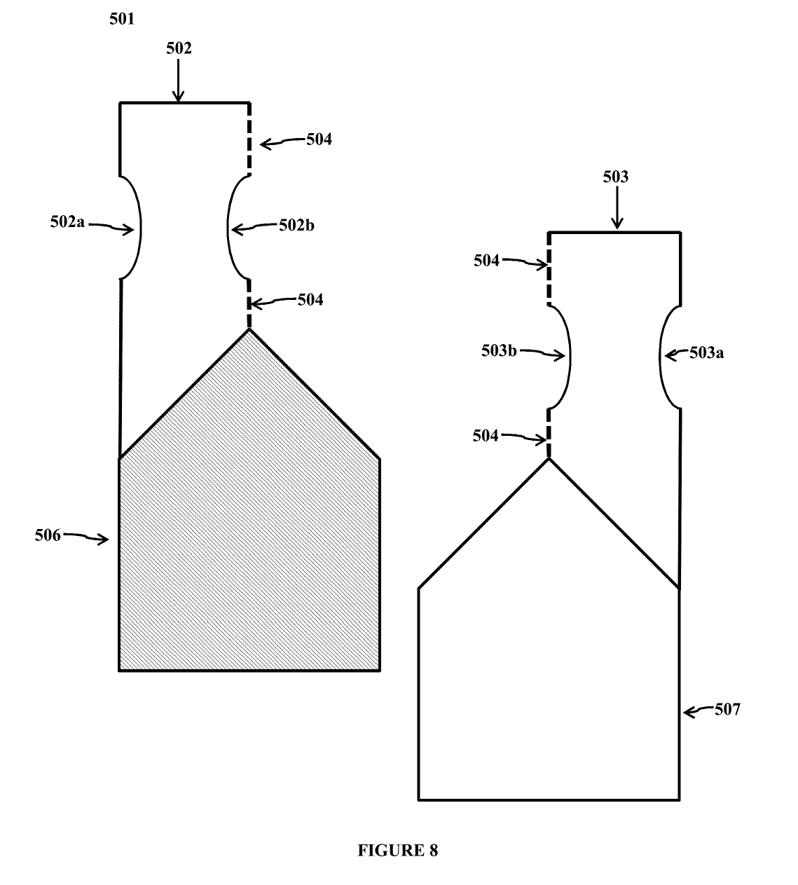

FIG. 8 shows the article of the invention 501 separated prior to overlaying the first flexible layer 507 on the second flexible layer 506. The left-tab 502 is attached to the second flexible layer 506. The right-tab 503 is attached to the first flexible layer 507. The second flexible layer 506 is placed beneath the first flexible layer 507 for sealing to create the enclosure that is of a size to hold the therapeutic as shown in FIG. 1. The left edge of the left-tab 502 further contains a cut-out 502a, and the right edge of the right-tab 503 contains a cut-out 503a to facilitate gripping of the article by the user. The left-tab 502 and right-tab 503 are fastened to each other at a longitudinal axis 504, which longitudinal axis is perforated. The left-tab 502 and right-tab 503 contain a cut-out at the right edge of the left-tab 502b and the left edge of the right-tab 503b along the longitudinal axis to facilitate separation of the left-tab 502 from the right-tab 503 as described above for FIG. 1.

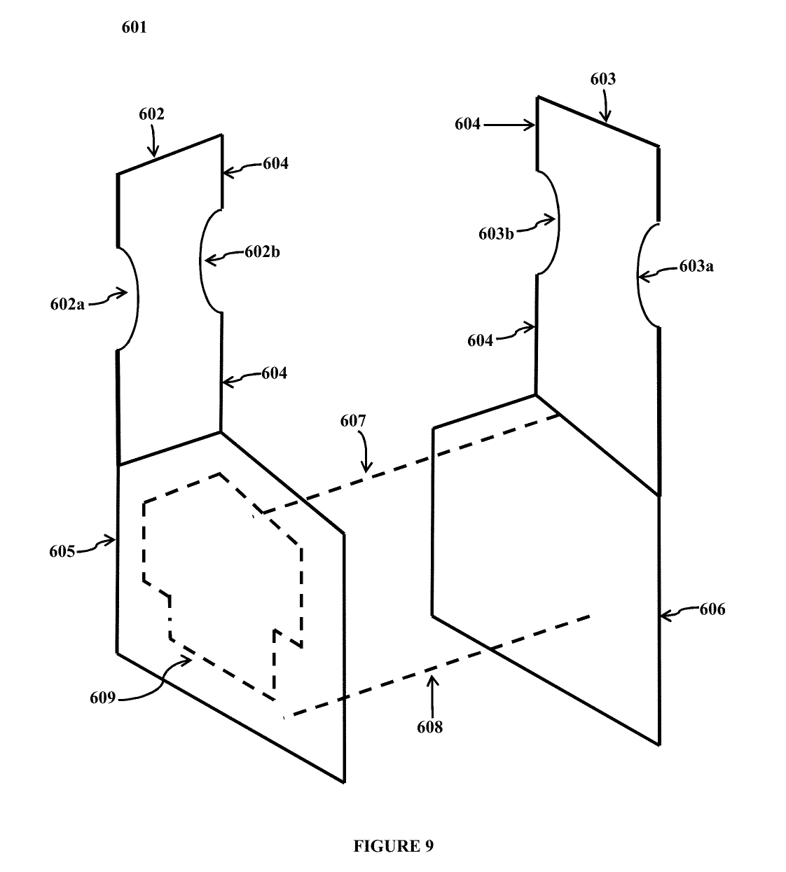

FIG. 9 shows the article of the invention 601 separated prior to overlaying the first flexible layer 606 on the second flexible layer 605. In this figure, the dashed lines 607 and 608 indicate how the left-tab 602 and the right-tab 603 are brought into contact and sealed with an adhesive to create the enclosure 609 of a size to hold the therapeutic. The left-tab 602 is attached to the second flexible layer 605. The right-tab 603 is attached to the first flexible layer 606. The second flexible layer 605 is placed beneath the first flexible layer 606 for sealing to create the enclosure 609 containing the therapeutic. The left edge of the left-tab 602 further contains a cut-out 602a, and the right edge of the right-tab 603 contains a cut-out 603a to facilitate gripping of the article by the user. The left-tab 602 and right-tab 603 are fastened to each other at a longitudinal axis 604, which longitudinal axis is perforated. The longitudinal axis can, for example, be a longitudinal median. The left-tab 602 and right-tab 603 contain a cut-out at the right edge of the left-tab 602b and the left edge of the right-tab 603b along the longitudinal axis to facilitate separation of the left-tab 602 from the right-tab 603 as described above for FIG. 1.

The length and width of an article of the invention can each independently be, for example, from about 5 mm to about 35 mm, from about 5 mm to about 25 mm, or from about 6 mm to about 21 mm. For example, the length can be about 21 mm and the width can be about 10 mm. The thickness of the article can be, for example, from about 0.5 mm to about 5 mm, from about 0.5 mm to about 3 mm, or from about 1 mm to about 2 mm. The width and depth of the enclosure containing the therapeutic in an article described herein can each independently be, for example, from about 1 mm to about 20 mm, from about 5 mm to about 15 mm, or from about 8 mm to about 12 mm. The height of the enclosure containing the therapeutic in an article described herein can be, for example, from about 0.5 mm to about 20 mm, from about 1 mm to about 10 mm, or from about 2 mm to about 4 mm.

### Method of Opening an Article Disclosed Herein.

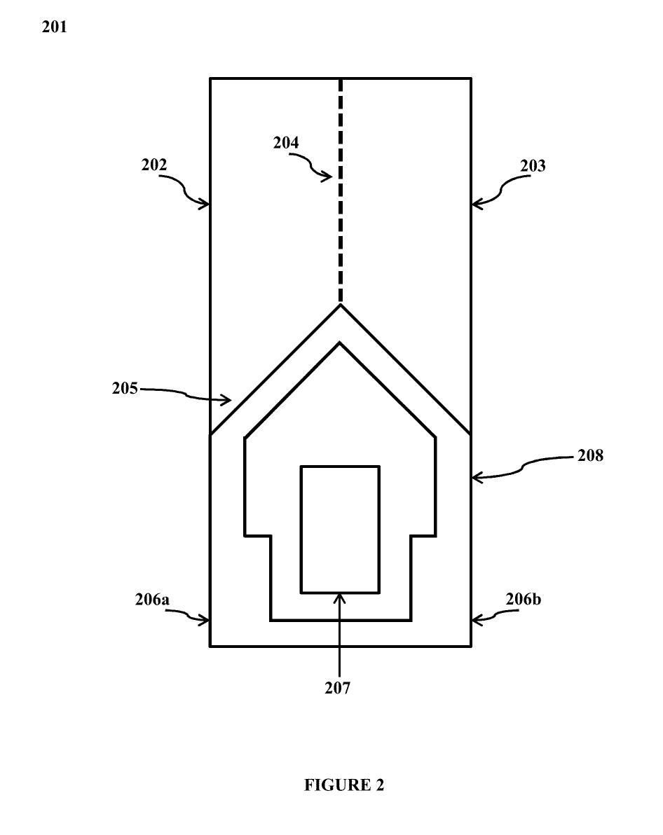

FIG. 2 depicts an illustrative example of an article 201 for storage of a unit dosage form of a therapeutic 207. The left-tab 202 and the right-tab 203 of the article 201 are separated by a perforated longitudinal axis 204 that can be separated by a subject. The left-tab 202 and the right-tab 203 are situated adjacent to each other in a common plane, and are pulled in opposing directions by the subject along the axis 204 so that the left-tab 202 and right-tab 203 are no longer in a common plane. The article is sealed 205 around the perimeter of an enclosure 208 using an adhesive, which enclosure 208 contains the unit dosage form of the therapeutic 207. The bottom of the article contains thicker seals 206a and 206b, which are wider than the seal 205 around the perimeter of the enclosure 208.

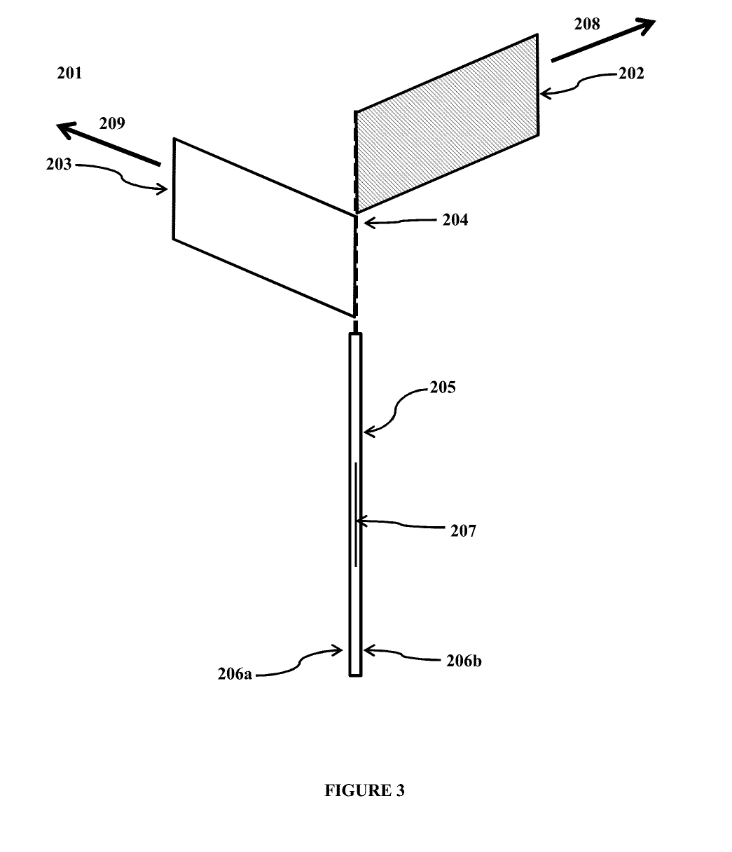

FIG. 3 shows a side view of the article 201 wherein the left-tab 202 and the right-tab 203 have been pulled apart as indicated by arrows 208 and 209 so that the left-tab 202 and right-tab 203 are no longer adjacent to each other, and are no longer in a common plane. The article 201 has been opened along the perforation, stopping at the top of the sealed perimeter 205 of the enclosure storing the unit dosage form of the therapeutic 207.

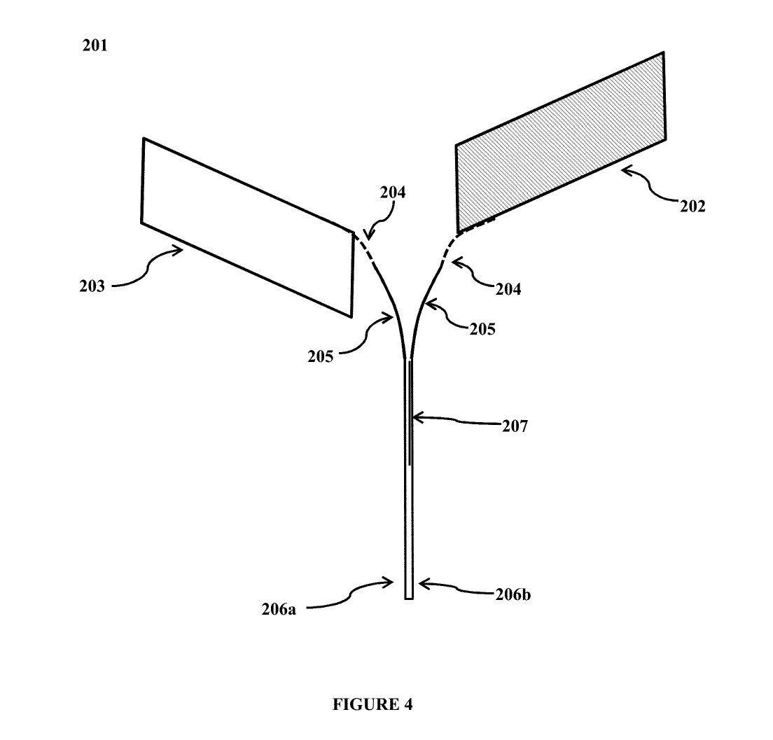

FIG. 4 shows another side view of the article 201, wherein the left-tab 202 and the right-tab 203 have been pulled apart enough to begin opening the seal 205 to render the unit dosage form of the therapeutic 207 accessible to the subject.

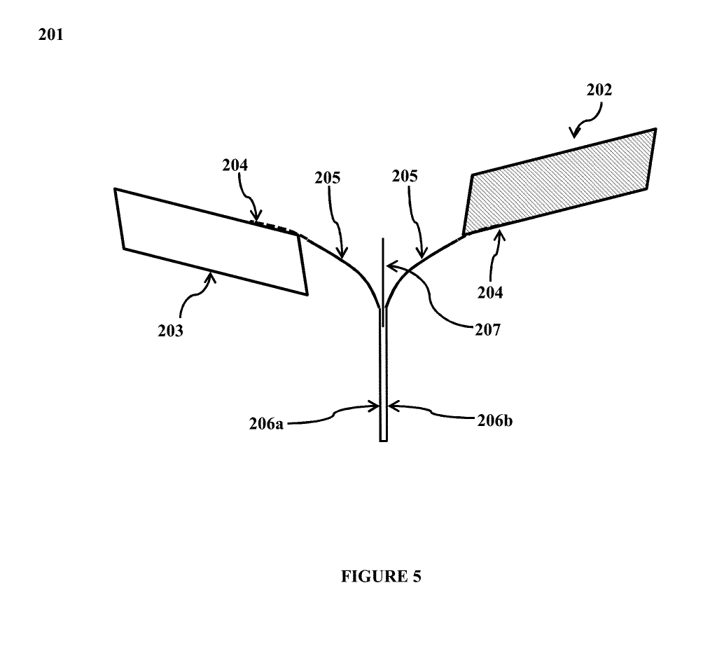

FIG. 5 shows the unsealing of the seal 205 around the perimeter of the enclosure until the seal reaches the wider, thicker seals 206a and 206b as shown in the front-view of the article in FIG. 1. The peeling open of the seal 205 around the perimeter of the enclosure provides an aperture that renders accessible the unit dosage form of the therapeutic 205 to the subject. The subject then transfers the therapeutic 207 directly from the article into the mouth.

### Placing a Unit Dosage Form of a Therapeutic in an Article of the Invention.

To place a unit dosage of a therapeutic in an article of the invention, first, an enclosure, which forms the bottom portion of the article for storage of a unit dosage form a therapeutic, is formed by overlaying a first flexible layer and a second flexible layer. The first flexible layer is attached to a right-tab of the article as shown in FIGS. 8 and 9. The second flexible layer is attached to a left-tab of the article as shown in FIGS. 8 and 9. Then, the first flexible layer and the second flexible layer are overlaid on each other, and sealed together around a perimeter using a peelable sealing adhesive, leaving one side of the enclosure open. The overlaying of the first flexible layer and the second flexible layer makes the left-tab and right-tab become adjacent to one another.

The unit dosage form of the therapeutic is inserted into the enclosure through the open side of the enclosure. The open side of the enclosure is then sealed using the same peelable adhesive as for the other sides of the enclosure.

The left-tab and the right-tab are sealed together along a perforation to become adjacent to each other along the perforated median and in a common plane as shown in FIGS. 8 and 9, and can be pulled in opposing directions by a subject.

Alternatively, the left-tab and the right-tab can be manufactured as a single layer of material, and perforated. In this case, the left-tab and the right-tab do not need to be sealed together after the enclosure is sealed.

### Physical Properties of an Article of the Invention.

Non-limiting examples of materials that can be used in the manufacture of, for example, the right-tab, left-tab, the enclosure, the first flexible layer, or the second flexible layer of the article include aluminum, foil, olefin fibers, paper, plastic, polyester, polyethylene fibers, polyethylene, and terephthalate. The aluminum can be an aluminum alloy containing, for example, chromium, copper, iron, magnesium, manganese, silicon, titanium, or zinc.

An article of the invention can be in the shape of, for example, a triangle, a square, a rectangle, a polygon, a pentagon, a hexagon, an arch, a curve, or any combination thereof. The portion of the article housing the therapeutic can be in the shape of, for example, a triangle, a square, a rectangle, a polygon, a pentagon, a hexagon, an arch, a curve, or any combination thereof. In some embodiments, an article of the invention is in the shape of a rectangle.

In some embodiments, the enclosure of a size to hold the therapeutic is in the shape of a pentagon. A first side of the pentagon and a second side of the pentagon can be about equal in length, a third side of the pentagon and a fourth side of the pentagon can be about equal in length, and a fifth side of the pentagon can be about perpendicular to the longitudinal axis and about parallel to the axis on which the two corners of the enclosure of the size to hold the unit dosage form of the therapeutic are disposed.

The third side of the pentagon and the fourth side of the pentagon can be about parallel to the longitudinal axis and about perpendicular to the axis on which the two corners of the enclosure of the size to hold the unit dosage form of the therapeutic are disposed. The first side of the pentagon and the second side of the pentagon can intersect at a vertex, and the vertex can be about collinear with the longitudinal axis. The seal along the perimeter of the enclosure of the size to hold the unit dosage form of the therapeutic can be weakest adjacent to the vertex.

An article of the invention can be sterilized via, for example, autoclaving, irradiation, hydrogen peroxide gas plasma, and ethylene oxide. Sterilization of the article can occur, for example, prior to insertion of the therapeutic in the article or prior to providing the article containing the therapeutic to a subject.

### Use of an Article of the Invention.

The present invention provides an article for storage of a therapeutic. The present invention further provides methods of opening an article described herein by a subject afflicted by a condition that affects, for example, the grip strength, muscle tone, or dexterity of the subject. The conditions can include, for example, amyotrophic lateral sclerosis (ALS), Huntington's disease, multiple sclerosis (MS), Parkinson's disease, primary lateral sclerosis, progressive bulbar palsy, progressive muscular atrophy, and pseudobulbar palsy. Loss of dexterity and decrease in grip strength can also occur or be comorbid with, for example, aging, brain damage, paralysis, trauma, or traumatic brain injury. In some embodiments, the condition treated herein is Parkinson's Disease.

An article of the invention can also be used to store those drugs that are sensitive to, for example, touch, or are hazardous when contacted with the skin. In some instances, degradation of a drug can occur when the drug comes into contact with skin. The degradation can occur when the drug is in the form of, for example, a pill, a capsule, a tablet, or a thin film. The present invention allows a user to directly transfer the therapeutic from the storage article to the mouth without any contact with the user's skin.

An article of the invention can be used to package drugs that can be delivered, for example, transdermally, orally, buccally, or sublingually.

Conditions that can be treated using a therapeutic delivered via, for example, a transdermal or sublingual film include, for example, allergic rhinitis, Alzheimer's Disease, angina pectoris, attention deficit hyperactivity disorder, benign prostatic hyperplasia, pain cardiac arrhythmia, depression, emesis, erectile dysfunction, hypertension, hypoglycemia, motion sickness, nicotine addiction, opioid dependence, pain, and Parkinson's Disease. Transdermal films can also be used, for example, for birth control, hormone replacement therapy, and vitamin delivery. In some embodiments, the subject has Parkinson's Disease.

Non-limiting examples of classes of therapeutics that can be packages, stored, or delivered, in an article of the invention include 5-alpha-reductase inhibitors, 5-aminosalicylates, 5HT3 receptor antagonists, ACE inhibitors, antivirals, cortical steroids, corticosteroid inhibitors, adrenergic bronchodilators, aldosterone receptor antagonists, alkylating agents, alpha-glucosidase inhibitors, amebicides, aminoglycosides, aminopenicillins, amino salicylates, AMPA receptor antagonists, amylin analogs, analgesics, androgens and anabolic steroids, angiotensin II inhibitors, angiotensin receptor blockers, antacids, anthelmintics, monoclonal antibodies, antiandrogens, antianginal agents, antiarrhythmic agents, antiasthmatic combinations, antibiotics, anti-emetics, bronchodilators, chronotropic agents, anticoagulants, anticonvulsants, antidepressants, antihistamines, antihyperlipidemic agents, antimalarial agents, antiseptic agents, sedatives, and hypnotics, aromatase inhibitors, barbiturates, BCR-ABL tyrosine kinase inhibitors, benzodiazepines, beta blockers, bile acid sequestrants, bisphosphonates, calcineurin inhibitors, calcitonin, calcium channel blocking agents, carbamate anticonvulsants, carbapenems, carbonic anhydrase inhibitors, catecholamines, cephalosporins, chelating agents, chemokine receptor antagonist, chloride channel activators, cholesterol absorption inhibitors, colony stimulating factors, contraceptives, corticotropin, coumarins and indandiones, COX-2 inhibitors, decongestants, dermatological agents, dipeptidyl peptidase 4 inhibitors, diuretics, echinocandins, EGFR inhibitors, estrogen receptor antagonists, estrogens, expectorants, Factor Xa inhibitors, gamma-aminobutyric acid reuptake inhibitors, GI stimulants, glucocorticoids, glucose elevating agents, glycylcyclines, gonadotropin releasing hormones, gonadotropin-releasing hormone antagonists, gonadotropins, growth hormones, H2 antagonists, heparin antagonists, heparins, HER2 inhibitors, histone deacetylase inhibitors, hormones, immune globulins, immunologic agents, immuno stimulants, immunosuppressive agents, impotence agents, insulin, ketolides, laxatives, leprostatics, leukotriene modifiers, lincomycin derivatives, lung surfactants, lysosomal enzymes, macrolides, mast cell stabilizers, meglitinides, metabolic agents, methylxanthines, mineralocorticoids, mitotic inhibitors, monoamine oxidase inhibitors, mTOR inhibitors, mucolytics, muscle relaxants, mydriatics, nicotinic acid derivatives, NK1 receptor antagonists, NNRTIs, nonsteroidal anti-inflammatory agents, NS5A inhibitors, nucleoside reverse transcriptase inhibitors (NRTIs), oral nutritional supplements, oxazolidinedione anticonvulsants, oxazolidinone antibiotics, parathyroid hormone and analogs, PCSK9 inhibitors, penicillins, peripheral vasodilators, phenothiazine antipsychotics, phosphate binders, plasma expanders, platelet aggregation inhibitors, platelet-stimulating agents, polyenes, probiotics, progesterone receptor modulators, progestins, prolactin inhibitors, prostaglandin D2 antagonists, protease inhibitors, protease-activated receptor-1 antagonists, proteasome inhibitors, proton pump inhibitors, psoralens, psychotherapeutic agents, purine nucleosides, quinolones, renin inhibitors, respiratory agents, rifamycin derivatives, salicylates, sclerosing agents, selective phosphodiesterase-4 inhibitors, selective serotonin reuptake inhibitors, serotonin-norepinephrine reuptake inhibitors, serotoninergic neuroenteric modulators, SGLT-2 inhibitors, smoking cessation agents, somatostatin and somatostatin analogs, statins, streptomyces derivatives, sulfonamides, sulfonylureas, synthetic ovulation stimulants, tetracyclines, thiazolidinediones, thioxanthenes, thrombin inhibitors, thyroid drugs, TNF-alpha inhibitors, vasodilators, vasopressin antagonists, vasopressors, VEGF/VEGFR inhibitors, vaccines, and vitamins.

Non-limiting examples of therapeutics that can be packaged, stored, or delivered, in an article of the invention include acebutolol, acetylcysteine, albuterol, alfentanil, allylprodine, alphaprodine, alprenolol, anileridine, apomorphine, aporphine, asprin, (−)-atenolol, azithromycin, benzylmorphine, betaxolol, bezitramide, bisoprolol, buprenorphine, butorphanol, carteolol, carvedilol, ceftriaxone, clonitazene, codeine, coumadin, desomorphine, dexpropranolol, dextromoramide, dezocine, diampromide, diamorphone, digoxin, dihydrocodeine, dihydromorphine, dimenoxadol, dimepheptanol, dimethylthiambutene, dioxaphetyl butyrate, dipipanone, enalapril, enoxaparin, eptazocine, ethoheptazine, ethylmethylthiambutene, ethylmorphine, etonitazene, fentanyl, furosemide, hydrocodone, hydromorphone, morphine, oxycodone, oxymorphone, guaifenesin, harmalol, heparin, heroin, hydralazine, hydrochlorothiazide, hydroxypethidine, ipratropium, isomethadone, isosorbide mononitrate, ketobemidone, labetalol, levobunolol, levofloxacin, levorphanol, levophenacylmorphan, lisinopril, lofentanil, losartan, lovenox, meperidine, meptazinol, metazocine, methadone, methylprednisolone, metopon, metoprolol, moxifloxacin, myrophine, nadolol, narceine, nicomorphine, norlevorphanol, normethadone, nalorphine, nalbuphene, norbuprenorphine, normal saline, normorphine, norpipanone, opium, oxprenolol, oxymorphone, papvereturn, penbutolol, pentazocine, phenadoxone, phenomorphan, phenazocine, phenoperidine, piminodine, pindolol, piritramide, practolol, propheptazine, propranolol, promedol, pronetalol, properidine, propoxyphene, R-atenolol, S-propranolol, sufentanyl, tapentadol, tilidine, timolol, torsemide, tramadol, sotalol, spironolactone, tiotropium, valsortan, vancomycin, zaroxolyn, zithromax, or a pharmaceutically-acceptable salt thereof of any of the foregoing. In some embodiments, an article of the invention is used to package, store, or deliver apomorphine, or a pharmaceutically-acceptable salt thereof, to a subject. In some embodiments, the therapeutic is apomorphine. In some embodiments, an article of the invention is used to store a thin-film that delivers apomorphine to a patient having Parkinson's Disease.

Non-limiting examples of materials that can be used for the thin-film include, for example, acacia gum, arabic gum, carboxymethyl cellulose, carboxyvinyl copolymers, cellulose acetate phthalate, ethyl cellulose, gelatin, hydrophilic polymers, hydroxypropyl methyl cellulose phthalate, hydroxypropyl ethyl cellulose, hydroxypropyl methyl cellulose, hydroxy propyl cellulose, guar gum, hydroxyethyl cellulose, maltodextrin, methylmethacrylate copolymer, pectin, polyacrylic acid, polyethylene oxide, polyethylene glycol, polymerized rosin, polyvinyl alcohol, polyvinyl pyrrolidone, pullulan, sodium alginate, sodium carboxy methyl cellulose, starch, tragancanth gum, xanthan gum.

Other polymers that can be used in a thin-film include, for example, poly(glycolic acid) (PGA), poly(lactic acid) (PLA), polydioxanoes, polyoxalates, poly(α-esters), polyanhydrides, polyacetates, polycaprolactones, poly(orthoesters), polyamino acids, polyaminocarbonates, polyurethanes, polycarbonates, polyamides, poly(alkyl cyanoacrylates), stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and caproic acid, copolymers of α-benzyl glutamate and polyethylene glycol, copolymers of succinate and poly(glycols), polyphosphazene, polyhydroxy-alkanoates, and mixtures thereof.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), each of which is incorporated by reference in its entirety.

Laminates can be used to protect, for example, thin-film drugs for oral delivery or transdermal patches from degradation or contamination. The laminate can be made of, for example, hydroxypropyl methylcellulose, latex, metal, metallic plastic, plastic, or polymer.

### Dosage Amounts.

In practicing the methods of treatment or use provided herein, therapeutically-effective amounts of the compounds described herein are administered in pharmaceutical compositions to a subject having a disease or condition to be treated. A therapeutically-effective amount can vary widely depending on the severity of the disease, the age and relative health of the subject, the potency of the compounds used, and other factors. Subjects can be, for example, humans, elderly adults, adults, adolescents, pre-adolescents, children, toddlers, infants, or neonates. A subject can be a patient.

A therapeutically-effective amount of a compound described herein can be present in an article of the invention in an amount of, for example, about 0.01 μg, about 0.05 μg, about 0.1 μg, about 0.15 μg, about 0.2 μg, about 0.25 μg, about 0.3 μg, about 0.35 μg, about 0.4 μg, about 0.5 μg, about 0.6 μg, about 0.7 μg, about 0.8 μg, about 0.9 μg, about 1 μg, about 2 μg, about 3 μg, about 4 μg, about 5 μg, about 10 μg, about 15 μg, about 20 μg, about 25 μg, about 30 μg, about 35 μg, about 40 μg, about 45 μg, about 50 μg, about 60 μg, about 70 μg, about 80 μg, about 90 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1 mg, about 2 mg, about 3 mg, about 4 mg, about 5 mg, about 6 mg, about 7 mg, about 8 mg, about 9 mg, or about 10 mg.

The size of the thin-film or transdermal film can correlate with the dosage of the therapeutic. For example, the size of the film can increase with dosage amount. The unit dosage form can be in the form of, for example, a thin-film or a transdermal patch. The length and width of the unit dosage form of the therapeutic can be from about 6 mm to about 21 mm, and the thickness can be, for example, less than 2 mm.

The article can be configured to be, for example, opened, peeled, or broken, by a subject. The subject can open the article by, for example, peeling apart the seal along the perimeter of the enclosure containing the therapeutic. The peeling apart of the seal along the perimeter of the enclosure can be facilitated by the pulling apart of the left-tab and the right-tab along the perforated longitudinal axis. The subject can pull apart the left-tab and the right-tab in opposing directions so that the two tabs are no longer in a common plane. The separation can also occur by the subject pulling apart the two tabs in opposing directions along a vertical plane so that the two parts remain in a common plane.

After opening of the article, the subject can remove the unit dosage form by, for example, pouring or transferring the unit dosage form out of the article into the subject's hand and placing the unit dosage form in the mouth or on the skin, as required. The subject can also remove the unit dosage form by, for example, pouring or transferring the therapeutic out of the article and directly into the mouth.

The wider seals 109a and 109b as shown in FIG. 1 can allow the article to remain sealed at the bottom of the article to create a cup that can hold the unit dosage form in place. The cup can allow the subject to consume the therapeutic by pouring the unit dosage form of the therapeutic directly into the mouth without needing to grip the unit dosage form by the fingers.

The unit dosage form of a therapeutic described herein can be administered before, during, or after the occurrence of a disease or condition, and the timing of administering the therapeutic can vary. For example, the therapeutic can be used as a prophylactic and can be administered continuously to subjects with a propensity to conditions or diseases in order to lessen or reduce a likelihood of the occurrence of the disease or condition. The therapeutic can be administered to a subject during or as soon as possible after the onset of the symptoms. The administration of the therapeutic can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any therapeutic described herein.

A therapeutic can be administered as soon as is practical after the onset of a disease or condition is detected or suspected, and for a length of time necessary for the treatment of the disease, such as, for example, from about 1 month to about 3 months. The length of time a compound can be administered can be, for example, about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Therapeutics described herein can be in unit dosage forms suitable for single administration of precise dosages. In the unit dosage form, the therapeutic is divided into unit doses containing appropriate quantities of one or more compounds. The unit dosage can be in the form of a package containing discrete quantities of the therapeutic. Non-limiting examples are thin-film strips or transdermal patches. Multiple-dose reclosable containers can be used, for example, in combination with or without a preservative.

An article of invention can be packaged as part of a kit. In some embodiments, a kit includes written instructions on the administration or use of the composition. The written material can be, for example, a label. The written material can suggest conditions and methods of administration. The instructions provide the subject and the supervising physician with the best guidance for achieving the optimal clinical outcome from the administration of the therapy. In some embodiments, the label can be approved by a regulatory agency, for example the U.S. Food and Drug Administration (FDA), the European Medicines Agency (EMA), or other regulatory agencies.

### Seal of an Article of the Invention.

An article of the invention can contain an enclosure that is sealed around the perimeter of the enclosure for storage of a therapeutic. An article disclosed herein can be configured to be sealed for storage of a therapeutic. The seal strength can indicate the integrity and resilience of the seal. Seal strength can be measured using, for example, the tensile seal strength, inflation seal strength, burst, and whole package creep testing methods. Seal strength can be tested and measured at the time a seal is formed, for example, after an article is sealed at a given sealing temperature and then cooled to room temperature. The tensile strength method can measure the seal strength of a defined-width sample of a package perimeter seal, which can be pulled apart by a moving jaw at a constant speed. The resistance force created during the seal separation can then be measured and represented as the seal strength. The inflation seal-strength test can be represented by either the burst or creep test. In the burst test, the whole article is inflated at a uniform rate until the article ruptures. The burst inflation method can measure the peak pressure at rupture to determine seal strength. In the creep test, the article is inflated to a constant pressure and the pressure is held either for a fixed time or until rupture occurs, and the time to rupture is measured.

A seal used in the article can be created by, for example, chemical or mechanical means. The seal can be made via, for example, heat-sealing, radio frequency wave sealing, cold sealing, adhesive sealing, or any combination thereof.

Cold sealing can use pressure-sensitive adhesives to seal the adhesive to the adherend. Non-limiting examples of pressure-sensitive adhesives include viscoelastic polymers, acrylic, natural rubber, synthetic rubber, silicone rubber, butyl rubber, nitriles, styrene block copolymers, vinyl ethers, and ethylene-vinyl-acetate.

Radio frequency wave sealing can be used to seal together two or more layers of film including, for example, PVC, EVA, and polyurethane. Radio frequency sealing can use radio waves directed through two or more layers of a dielectric plastic in conjunction with pressure to cause the molecules of all layers of the plastic to combine when the material becomes molten. The changing polarity of the radio waves being passed through the plastic can cause polarized molecules in the polymer to vibrate back and forth. The vibration can induce friction at the molecular level and produce heat. This heat can cause the plastic to melt, and, under pressure, the layers can seal together by free exchange of molecules. After the seal has been created, the article is allowed to cool to room temperature.

Non-limiting examples of adhesives that can be used in an article of the invention include, ABS (acrylonitrile-butadiene-styrene), acetal, aclar, acrylic, acrylonitrile-methyl acrylate copolymer, APET, butyrate, cellulose acetate, cellulose acetate butyrate, cellulose triacetate, cellulose nitrate, CPET, epoxy, ethyl cellulose, EVA (ethyl vinyl acetate), EVOH (ethyl vinyl alcohol), fluorinated ethylene-propylene copolymer, hydroxyethyl cellulose, methyl cellulose, nylon (polyamide), pellethane, PET (polyethylene terephthalate), PETG (polyethylene terephthalate glycol), phenol-formaldehyde, polycarbonate, polyethylene, polyethylene resin, polyester-polyurethane resin, polyol-polyurethane resin, polyurethane, polyurethane foam, polyvinyl butyral, polyvinylidene chloride, vinyl chloride-acetate, polyethylene glycol, polyimide, polymethyl methacrylate, polypropylene, polystyrene, polyvinyl acetate, polyvinyl acetate-chloride copolymer polyvinyl chloride, polyvinyl acetal, polyvinylidene chloride, polyvinyl formal, polyalkylene terephthalate, polyvinyl alcohol, rubber, silicone, sodium carboxymethyl, tetrafluoroethylene, and vinyl resin.

The size of the seal can correlate with the strength of the seal. For example, a wider seal can create more resistance for a subject in opening of the seal, and a narrow seal can allow for easier peeling of the seal compared to a wider seal. The seal can also contain, for example, dimples, indentations, clefts, or depressions to create additional resistance, and increase the strength of the seal.

### Method of Manufacturing an Article of the Invention.

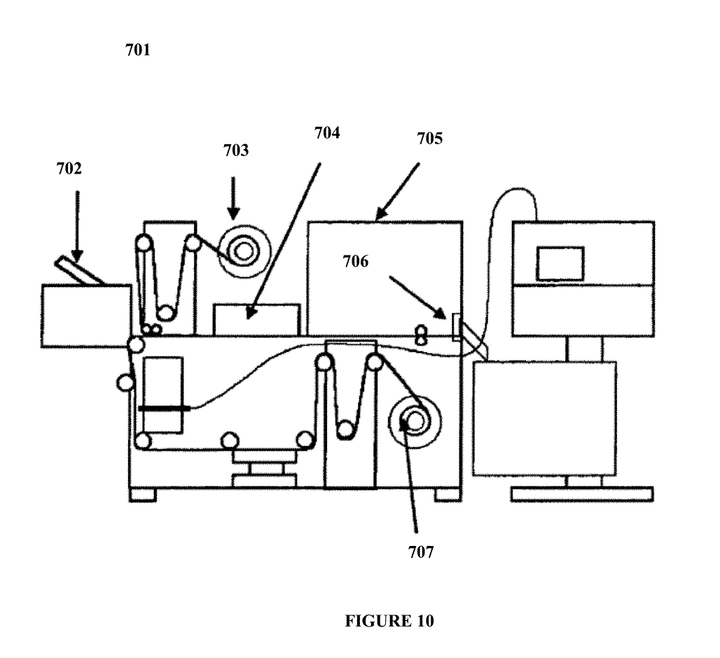

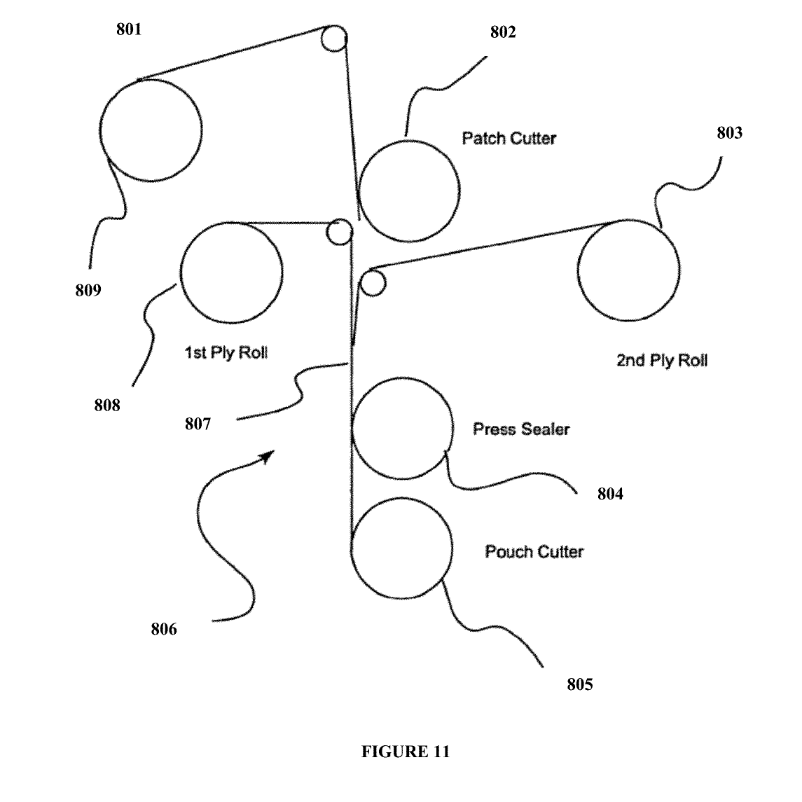

FIGS. 10 and 11 depict machines 701 and 801, respectively, which can be used for packaging a thin-film into an article of an invention as described herein. The machines can operate intermittently or continuously, and can be oriented horizontally or vertically. For example, the mechanism 705 in FIG. 10 operates intermittently and is oriented horizontally, while the mechanism 806 in FIG. 11 operates continuously and is oriented vertically.

A method for packaging a thin-film can use the machines of FIGS. 10 and 11. For example, the method can include placing the thin-film between the first flexible layer and second flexible layer as described above. The first flexible layer and the second flexible layer can be provided in continuous rolls of stock materials as shown in the first and second rolls 703 and 707 in FIGS. 10 and 808 and 803 in FIG. 11, which rolls are disposed on payouts of the machines 701 and 801. The rolls of material can provide webs 807 as shown in FIG. 11 between which, or upon which, the thin-films can be disposed so that the patches are placed between the first and second flexible layers. The rolls of material can be sized larger or wider than the article of the invention so that more than one article can be produced simultaneously by placing numerous thin-films between the flexible layers.

FIG. 10 shows that the packaging can be oriented horizontally with the webbing oriented horizontally so that the thin-films can be disposed onto one of the flexible layers. Thus, one of the rolls or payouts can be a lower roll or payout 707, while the other is an upper roll or payout 703.

Alternatively, in FIG. 11, the packaging can be oriented vertically with the webbing oriented vertically so that the thin-films can be disposed between the flexible layers.

FIG. 10 shows that the thin-films can be provided stacked in a cartridge 702 adjacent to the webbing. One or more thin-films can be picked from the cartridge and placed on the web.

Alternatively, in FIG. 11, the thin-films can be provided in a continuous roll 809 disposed on a payout of the machine. Individual thin-films can be separated or cut from the roll prior to the webbing, and placed on the webbing. In FIG. 11, the mechanism can include a cutter with blades shaped to form the thin-films. The cutter can be a rolling cutter 802 with a cylindrical shape that makes rolling contact with the web to continuously cut or separate the thin-films from the web.

After the thin-films have been placed between the flexible layers of the article, at least a portion of the flexible layers are sealed together to form enclosures around the thin-films. For example, a seal entirely seals the thin-film in the enclosure. The flexible layers can be sealed by applying heat and pressure against the flexible layers for a predetermined time period. The mechanisms can include a heat seal station 704 as shown in FIG. 10 in which a press is movably disposed to contact the flexible layers selectively. The press 704 can be shaped and sized similar to a mold or blank of the seal. Thus, when the press 704 contacts the flexible layers, the seal is formed in the desired shape and size. The press 704 can be further configured to form numerous seals at the same time.

As shown in FIG. 10, the press 704 can be applied against the flexible layers in a linear motion. Thus, the heat and pressure can be applied intermittently by the press. Alternatively, as shown in FIG. 11, the press 804 can be cylindrical and coupled to the mechanism to make rotating contact with the flexible layers. Thus, the heat and pressure can be applied continuously, and the webbing can be run continuously. The press can have circular or arc-shaped molds or blanks of the seal.

Portions of the flexible layers that extend beyond the seal or enclosure can be left unsealed to form the tabs of an article of the invention. The numerous pouches can be separated from one another and the webbing to form individual pouches with individual patches therein. The mechanisms can include a shearing station for separating or cutting individual pouches from the webbing. As shown in FIG. 10, the mechanism can include blades 706 that are shaped and sized to cut the articles from the webbing. The blades can include numerous blades to cut numerous pouches simultaneously. The blades can contact the webbing or plies linearly. Thus, the blades can operate intermittently.

Alternatively, as shown in FIG. 11, the mechanism can include a cylindrical blade 805 that is coupled to the machine to make rolling contact with the webbing. Thus, the cylindrical blade can continuously cut the articles from the webbing.

### EMBODIMENTS

The following non-limiting embodiments provide illustrative examples of the invention, but do not limit the scope of the invention.

### Embodiment 1

An article for storage of a therapeutic, the article comprising: a) a first flexible layer, wherein the first flexible layer comprises a right-tab and a backing layer; and b) a second flexible layer, wherein the second flexible layer comprises a left-tab and a cover layer, wherein the right-tab and the left-tab are adjacent and connected to each other along an axis, wherein the right-tab and the left-tab are in a common plane, and wherein the axis is weakened in comparison to the right-tab and the left-tab; and wherein the cover layer is overlaid on the backing layer to create an enclosure of a size to hold a unit dosage form of the therapeutic, wherein the cover layer and backing layer are sealed together along a perimeter of the enclosure, wherein a portion of the perimeter of the enclosure is adjacent to an end of the axis.

### Embodiment 2

The article of embodiment 1, wherein the seal along the perimeter of the enclosure of the size to hold the unit dosage form of the therapeutic is weakest about directly adjacent to the axis.

### Embodiment 3

The article of any one of embodiments 1-2, wherein the portion of the perimeter of the enclosure adjacent to the end of the axis is a vertex.

### Embodiment 4

The article of any one of embodiments 1-3, wherein the portion of the perimeter of the enclosure adjacent to the end of the axis is a top of a curve.

### Embodiment 5

The article of any one of embodiments 1-4, wherein the axis is about longitudinal.

### Embodiment 6

The article of embodiment 5, wherein the enclosure comprises two corners that form a side that is about perpendicular to the axis.

### Embodiment 7

The article of any one of embodiments 1-6, wherein the seal along the perimeter of the enclosure is strongest at an edge furthest from the portion of the perimeter of the enclosure adjacent to the end of the axis.

### Embodiment 8

The article of any one of embodiments 1-7, wherein the seal is of a strength to be broken by an elderly subject.

### Embodiment 9

The article of any one of embodiments 1-8, wherein the seal is of a strength to be broken by a subject with reduced dexterity.

### Embodiment 10

The article of any one of embodiments 1-9, wherein the enclosure of the size to hold the unit dosage form of the therapeutic has a cup shape.

### Embodiment 11

The article of any one of embodiments 1-10, wherein the enclosure of the size to hold the unit dosage form of the therapeutic has a height that is from about 0.1 mm to about 20 mm.

### Embodiment 12

The article of any one of embodiments 1-11, wherein the axis is perforated.

### Embodiment 13

The article of any one of embodiments 1-12, wherein the enclosure of the size to hold the unit dosage form of the therapeutic is pentagonal in shape.

### Embodiment 14

A method for storage of a therapeutic, the method comprising: a) inserting a unit dosage form of the therapeutic in a cavity; and b) sealing the cavity along a perimeter of the cavity to transform the cavity into an enclosure of size to hold the unit dosage form of the therapeutic; wherein the enclosure forms part of an article for storage of the therapeutic, wherein the article comprises: i) a first flexible layer, wherein the first flexible layer comprises a right-tab and a backing layer; and ii) a second flexible layer, wherein the second flexible layer comprises a left-tab and a cover layer, wherein the right-tab and the left-tab are adjacent and fastened to each other along an axis, wherein the right-tab and the left-tab are in a common plane, and wherein the fastening of the axis is weakened with respect to the right-tab and the left-tab; and wherein the cover layer is overlaid on the backing layer to create the enclosure of the size to hold the unit dosage form of the therapeutic, wherein the cover layer and backing layer are sealed together along a perimeter of the enclosure, wherein a portion of the perimeter of the enclosure is adjacent to an end of the axis.

### Embodiment 15

The method of embodiment 14, wherein the axis is about longitudinal.

### Embodiment 16

A method of opening an article for storage of a therapeutic by a subject, the method comprising: a) gripping a left-tab and a right-tab of the article, wherein the article comprises: i) a first flexible layer, wherein the first flexible layer comprises the right-tab and a backing layer; and ii) a second flexible layer, wherein the second flexible layer comprises the left-tab and a cover layer, wherein the right-tab and the left-tab are adjacent and fastened to each other along an axis, wherein the right-tab and the left-tab are in a common plane, and wherein the fastening of the axis is weakened with respect to the right-tab and the left-tab; and wherein the cover layer is overlaid on the backing layer to create an enclosure of a size to hold a unit dosage form of the therapeutic, wherein the cover layer and backing layer are sealed together along a perimeter of the enclosure, wherein a portion of the perimeter of the enclosure is adjacent to an end of the axis; b) pulling apart the left-tab and the right-tab so that the left-tab and right-tab are no longer adjacent to each other; and c) peeling open at least part of the seal of the enclosure of the size to hold the unit dosage form of the therapeutic using the left-tab and the right-tab; wherein the peeling open of the enclosure provides an aperture that renders accessible the unit dosage form of the therapeutic to the subject.

### Embodiment 17

The method of embodiment 16, wherein the left-tab and the right-tab are no longer in the common plane upon the pulling apart of the left-tab and the right-tab.

### Embodiment 18

The method of any one of embodiments 16-17, further comprising removing of the unit dosage form of the therapeutic by transferring the unit dosage form of the therapeutic through the aperture to the mouth of the subject.

### Embodiment 19

The method of any one of embodiments 16-18, wherein the axis is about longitudinal.

### Embodiment 20

A kit comprising: a) a package, wherein the package comprises: i) a first flexible layer, wherein the first flexible layer comprises a right-tab and a backing layer; and ii) a second flexible layer, wherein the second flexible layer comprises a left-tab and a cover layer, wherein the right-tab and the left-tab are adjacent and fastened to each other along an axis, wherein the right-tab and the left-tab are in a common plane, and wherein the fastening of the axis is weakened with respect to the right-tab and the left-tab; and wherein the cover layer is overlaid on the backing layer to create an enclosure of a size to hold a unit dosage form of a therapeutic, wherein the cover layer and backing layer are sealed together along a perimeter of the enclosure, wherein a portion of the perimeter of the enclosure is adjacent to an end of the axis; and b) a therapeutic enclosed in the enclosure.

### Embodiment 21

The kit of embodiment 20, wherein the unit dosage form is in the form of a film.

### Embodiment 22

The kit of any one of embodiments 20-21, wherein the unit dosage form is in the form of a strip.

### Embodiment 23

The kit of any one of embodiments 20-22, further comprising written instructions for opening of the package.

### Embodiment 24

The kit of any one of embodiments 20-23, wherein the axis about longitudinal.

### Embodiment 25

A method of treating a condition, the method comprising administering to a subject in need thereof a unit dosage form of a therapeutic, wherein the unit dosage form of the therapeutic is housed in an article for storage of the unit dosage form of the therapeutic, wherein the article for storage of the unit dosage form of the therapeutic is opened by the subject, wherein the opening of the article for storage of the unit dosage form of the therapeutic by the subject comprises: a) gripping a left-tab and a right-tab of the article by the subject, wherein the article comprises: i) a first flexible layer, wherein the first flexible layer comprises the right-tab and a backing layer; and ii) a second flexible layer, wherein the second flexible layer comprises the left-tab and a cover layer, wherein the right-tab and the left-tab are adjacent and fastened to each other along an axis, wherein the right-tab and the left-tab are in a common plane, and wherein the fastening of the axis is weakened with respect to the right-tab and the left-tab; and wherein the cover layer is overlaid on the backing layer to create an enclosure of a size to hold the unit dosage form of the therapeutic, wherein the cover layer and backing layer are sealed together along a perimeter of the enclosure, wherein a portion of the perimeter of the enclosure is adjacent to an end of the axis; b) pulling apart the left-tab and the right-tab so that the left-tab and right-tab are no longer adjacent to each other; c) peeling open at least part of the seal of the enclosure of the size to hold the unit dosage form of the therapeutic using the left-tab and the right-tab; wherein the peeling open of the enclosure provides an aperture that renders accessible the unit dosage form of the therapeutic to the subject; and d) removing the unit dosage form of the therapeutic by transferring the unit dosage form of the therapeutic through the aperture to the mouth of the subject.

### Embodiment 26

The method of embodiment 25, wherein the condition is Parkinson's Disease.

### Embodiment 27

The method of any one of embodiments 25-26, wherein the unit dosage form is in the form of a film.

### Embodiment 28

The method of any one of embodiments 25-27, wherein the unit dosage form is in the form of a strip.

### Embodiment 29

The method of any one of embodiments 25-28, wherein the therapeutic is apomorphine, or a pharmaceutically-acceptable salt thereof.

### Embodiment 30

The method of any one of embodiments 25-29, wherein the axis is about longitudinal.

What is claimed is:

1. An article for storage of a therapeutic, the article comprising:
a) a first flexible layer, wherein the first flexible layer comprises a right-tab and a backing layer; and
b) a second flexible layer, wherein the second flexible layer comprises a left-tab and a cover layer,
wherein the right-tab and the left-tab are adjacent and connected to each other along an axis, wherein the right-tab and the left-tab are in a common plane, and wherein the axis is weakened in comparison to the right-tab and the left-tab; and
wherein the cover layer is overlaid on the backing layer to create an enclosure of a size to hold a unit dosage form of the therapeutic, wherein the cover layer and backing layer are sealed together along a perimeter of the enclosure, wherein a portion of the perimeter of the enclosure is adjacent to an end of the axis.

2. The article of claim 1, wherein the seal along the perimeter of the enclosure of the size to hold the unit dosage form of the therapeutic is weakest about directly adjacent to the axis.

3. The article of claim 1, wherein the portion of the perimeter of the enclosure adjacent to the end of the axis is a vertex.

4. The article of claim 1, wherein the portion of the perimeter of the enclosure adjacent to the end of the axis is a top of a curve.

5. The article of claim 1, wherein the axis is about longitudinal.

6. The article of claim 5, wherein the enclosure comprises two corners that form a side that is about perpendicular to the axis.

7. The article of claim 1, wherein the seal along the perimeter of the enclosure is strongest at an edge furthest from the portion of the perimeter of the enclosure adjacent to the end of the axis.

8. The article of claim 1, wherein the seal is of a strength to be broken by an elderly subject.

9. The article of claim 1, wherein the seal is of a strength to be broken by a subject with reduced dexterity.

10. The article of claim 1, wherein the enclosure of the size to hold the unit dosage form of the therapeutic has a cup shape.

11. The article of claim 1, wherein the enclosure of the size to hold the unit dosage form of the therapeutic has a height that is from about 0.1 mm to about 20 mm.

12. The article of claim 1, wherein the axis is perforated.

13. The article of claim 1, wherein the enclosure of the size to hold the unit dosage form of the therapeutic is pentagonal in shape.

14. A method for storage of a therapeutic, the method comprising:
a) inserting a unit dosage form of the therapeutic in a cavity; and
b) sealing the cavity along a perimeter of the cavity to transform the cavity into an enclosure of size to hold the unit dosage form of the therapeutic;
wherein the enclosure forms part of an article for storage of the therapeutic, wherein the article comprises:
i) a first flexible layer, wherein the first flexible layer comprises a right-tab and a backing layer; and
ii) a second flexible layer, wherein the second flexible layer comprises a left-tab and a cover layer,
wherein the right-tab and the left-tab are adjacent and fastened to each other along an axis, wherein the right-tab and the left-tab are in a common plane, and wherein the fastening of the axis is weakened with respect to the right-tab and the left-tab; and
wherein the cover layer is overlaid on the backing layer to create the enclosure of the size to hold the unit dosage form of the therapeutic, wherein the cover layer and backing layer are sealed together along a perimeter of the enclosure, wherein a portion of the perimeter of the enclosure is adjacent to an end of the axis.

15. The method of claim 14, wherein the axis is about longitudinal.

16. A method of opening an article for storage of a therapeutic by a subject, the method comprising:
a) gripping a left-tab and a right-tab of the article, wherein the article comprises:
i) a first flexible layer, wherein the first flexible layer comprises the right-tab and a backing layer; and
ii) a second flexible layer, wherein the second flexible layer comprises the left-tab and a cover layer,
wherein the right-tab and the left-tab are adjacent and fastened to each other along an axis, wherein the right-tab and the left-tab are in a common plane, and wherein the fastening of the axis is weakened with respect to the right-tab and the left-tab; and
wherein the cover layer is overlaid on the backing layer to create an enclosure of a size to hold a unit dosage form of the therapeutic, wherein the cover layer and backing layer are sealed together along a perimeter of the enclosure, wherein a portion of the perimeter of the enclosure is adjacent to an end of the axis;
b) pulling apart the left-tab and the right-tab so that the left-tab and right-tab are no longer adjacent to each other; and
c) peeling open at least part of the seal of the enclosure of the size to hold the unit dosage form of the therapeutic using the left-tab and the right-tab;
wherein the peeling open of the enclosure provides an aperture that renders accessible the unit dosage form of the therapeutic to the subject.

17. The method of claim 16, wherein the left-tab and the right-tab are no longer in the common plane upon the pulling apart of the left-tab and the right-tab.

18. The method of claim 16, further comprising removing of the unit dosage form of the therapeutic by transferring the unit dosage form of the therapeutic through the aperture to the mouth of the subject.

19. The method of claim 16, wherein the axis is about longitudinal.

20. A kit comprising:
a) a package, wherein the package comprises:
i) a first flexible layer, wherein the first flexible layer comprises a right-tab and a backing layer; and
ii) a second flexible layer, wherein the second flexible layer comprises a left-tab and a cover layer,
wherein the right-tab and the left-tab are adjacent and fastened to each other along an axis, wherein the right-tab and the left-tab are in a common plane, and wherein the fastening of the axis is weakened with respect to the right-tab and the left-tab; and
wherein the cover layer is overlaid on the backing layer to create an enclosure of a size to hold a unit dosage form of a therapeutic, wherein the cover layer and backing layer are sealed together along a perimeter of the enclosure, wherein a portion of the perimeter of the enclosure is adjacent to an end of the axis; and
b) a therapeutic enclosed in the enclosure.

21. The kit of claim 20, wherein the unit dosage form is in the form of a film.

22. The kit of claim 20, wherein the unit dosage form is in the form of a strip.

23. The kit of claim 20, further comprising written instructions for opening of the package.

24. The kit of claim 20, wherein the axis about longitudinal.

25. A method of treating a condition, the method comprising administering to a subject in need thereof a unit dosage form of a therapeutic, wherein the unit dosage form of the therapeutic is housed in an article for storage of the unit dosage form of the therapeutic, wherein the article for storage of the unit dosage form of the therapeutic is opened by the subject,
wherein the opening of the article for storage of the unit dosage form of the therapeutic by the subject comprises:
a) gripping a left-tab and a right-tab of the article by the subject, wherein the article comprises:
i) a first flexible layer, wherein the first flexible layer comprises the right-tab and a backing layer; and
ii) a second flexible layer, wherein the second flexible layer comprises the left-tab and a cover layer,
wherein the right-tab and the left-tab are adjacent and fastened to each other along an axis, wherein the right-tab and the left-tab are in a common plane, and wherein the fastening of the axis is weakened with respect to the right-tab and the left-tab; and
wherein the cover layer is overlaid on the backing layer to create an enclosure of a size to hold the unit dosage form of the therapeutic, wherein the cover layer and backing layer are sealed together along a perimeter of the enclosure, wherein a portion of the perimeter of the enclosure is adjacent to an end of the axis;
b) pulling apart the left-tab and the right-tab so that the left-tab and right-tab are no longer adjacent to each other;
c) peeling open at least part of the seal of the enclosure of the size to hold the unit dosage form of the therapeutic using the left-tab and the right-tab;
wherein the peeling open of the enclosure provides an aperture that renders accessible the unit dosage form of the therapeutic to the subject; and
d) removing the unit dosage form of the therapeutic by transferring the unit dosage form of the therapeutic through the aperture to the mouth of the subject.

26. The method of claim 25, wherein the condition is Parkinson's Disease.

27. The method of claim 25, wherein the unit dosage form is in the form of a film.

28. The method of claim 25, wherein the unit dosage form is in the form of a strip.

29. The method of claim 25, wherein the therapeutic is apomorphine, or a pharmaceutically-acceptable salt thereof.

30. The method of claim 25, wherein the axis is about longitudinal.
